(19) United States
(12) Patent Application Publication
(10) Pub. No.: US 2005/0181036 A1
(43) Pub. Date: Aug. 18, 2005

(54) Aerosol delivery of curcumin

(75) Inventors: Bharat B. Aggarwal, Houston, TX (US); Jack V. Knight, Houston, TX (US)

Correspondence Address: FULBRIGHT & JAWORSKI L.L.P., 600 CONGRESS AVE., SUITE 2400, AUSTIN, TX, 78701, US

(73) Assignee: Research Development Foundation

(21) Appl. No.: 10/925,731

(22) Filed: Aug. 25, 2004

Related U.S. Application Data
(60) Provisional application No. 60/498,135, filed on Aug. 26, 2003.

(51) Int. Cl. A61K 9/127; A61K 31/12

(52) U.S. Cl. 424/450; 514/689

(57) ABSTRACT

Pharmaceutical compositions suitable for aerosol delivery to a subject that include curcumin dispersed in a lipid vehicle, wherein the lipid has a transition temperature of less than about 15° C., are disclosed. In addition, methods of treating a pathological condition in a subject that include providing one of the claimed pharmaceutical compositions and administering the composition to the subject are disclosed. For example, the pathological condition can be a hyperproliferative disease, such as cancer, an inflammatory disease, or a pulmonary disease.

### CROSS-REFERENCE TO RELATED APPLICATIONS

This application claims priority to U.S. Provisional Application No. 60/498,135 filed on Aug. 26, 2003, which is incorporated herein by reference in its entirety.

### TECHNICAL FIELD

The present invention relates generally to the fields of pharmacology, lipid chemistry, and pathology. More particularly, it pertains to pharmaceutical compositions suitable for aerosol delivery to a subject and methods of treating a pathological condition in a subject using curcumin.

### BACKGROUND OF THE INVENTION

Curcumin (diferuloylmethane) is a natural yellow orange dye derived from the rhizome of Curcuma longa Linn, an East Indian plant. It is used as a spice and has been known as an Ayurvedic medicine for centuries on the Indian subcontinent. This normutritive phytochemical is pharmacologically safe, considering that it has been consumed daily as a dietary spice, at doses up to 100 mg/day, for centuries (Ammon et al., 1991). Recent phase I clinical trials indicate that people tolerate curcumin at a dose as high as 8 g/day (Cheng et al., 2001).

In the United States, curcumin is used as a coloring agent in cheese, spices, mustard, cereals, pickles, potato flakes, soups, ice creams, and yogurts. Curcumin is not water-soluble, but is soluble in ethanol or in dimethylsulfoxide. Numerous studies indicate that it has antioxidant and anti-inflammatory properties.

Curcumin has been shown to have a wide variety of clinical effects. Perhaps curcumin's best known therapeutic effect is its anti-cancer effect (see, e.g., Kuttan et al., 1985). In vivo studies in mice demonstrate that curcumin suppresses carcinogenesis of the skin (Conney et al., 1991; Huang et al., 1991; Lu et al., 1994; Limtrakul et al., 1997; Huang et al., 1997), the forestomach (Huang et al., 1994; Singh et al., 1998), the colon (Rao et al, 1995; Kim et al., 1998; Kawamori et al., 1999), and the liver (Chuang et al., 2000). Curcumin has been shown to inhibit the proliferation of a wide variety of tumor cells, including B cell and T cell leukemia (Kuo et al., 1996; Ranjan et al., 1999; Piwocka et al., 1999; Han et al., 1999), colon carcinoma (Chen et al., 1999), epidermoid carcinoma (Korutla and Kumar, 1994) and breast cancer (Aggarwal et al., 2003D).

Curcumin also has been implicated in the down regulation of the activation of nuclear factor-κB (NF-κB). This factor is activated in response to inflammatory stimuli, carcinogens, tumor promoters, and hypoxia, each of which frequently occurs in tumor cells (Pahl et al., 1999).

Curcumin has been implicated in a wide variety of other anticancer mechanisms. It has been shown to down regulate the activation of activator protein-1 (AP-1) (Huang et al., 1991) and c-Jun N-terminal kinase (JNK) (Chen and Tan, 1998), two transcription factors involved in cellular transformation. It has been implicated in the suppression of tumor cell adhesion to endothelial cells (Kumar et al., 1998) by down regulation of NF-KB activation (Kumar et al., 1998). It also down regulates cyclooxygenase-2 (COX-2) expression (Plummer et al, 1999; Zhang et al., 1999), mostly through down regulation of NF-KB. It has been shown to suppress angiogenesis in vivo (Mohan et al., 2000; Arbiser et al., 1998). It has been demonstrated to down regulate iNOS expression (Pan et al., 2000) and cyclin D1 expression (Bharti et al., 2000; Mukhopadhyay et al., 2002).

Curcumin has also been implicated to have a role in the pathogenesis of atherosclerosis and myocardial infarction. In particular, it has been found to inhibit the proliferation of vascular smooth muscle cells (Huang et al., 1992; Chen et al., 1998), which is a hallmark feature of atherosclerosis. There is evidence that it lowers serum cholesterol levels (Soni et al., 1992; Hussain and Chandrasekhara, 1992; Hussain and Chandrasekhara, 1994) and inhibits LDL oxidation (Quiles et al., 1998; Ramirez-Tortosa et al., 1999; Asai and Miyazawa, 2001; Naidu and Thippeswamy, 2002). It also inhibits platelet activation (Srivastava et al., 1985; Srivastava et al., 1986; Srivastava et al., 1995), and helps prevent myocardial ischemia after induction of experimentally-induced myocardial ischemia in the cat and rat (Dikshit et al., 1995; Nirmala and Puvanakrishnan, 1996; Nirmala and Puvanakrishnan, 1996; Nirmala et al., 1999). Curcumin has also been implicated in the suppression of diabetes (Arun and Nalini, 2002), and has also been found to stimulate muscle regeneration (Thaloor et al., 1999) and enhance wound healing (Sidhu et al., 1998) in animals. In addition, it has been found to suppress symptoms associated with rheumatoid arthritis (Deodhar, 1980) and reduce the incidence of cholesterol gall stone formation (Hussain and Chandrasekhara, 1992). Studies in mice have shown that curcumin significantly reduced the duration and severity of experimental allergic encephalomyelitis (EAE), thus implicating an effect of curcumin in the pathogenesis of multiple sclerosis (Natarajan and Bright, 2002). It has also been found to be a potent and selective inhibitor of HIV-1 long terminal repeat-directed gene expression, at concentrations that have minor effects on cells (Li et al., 1993). Lim et al. (2001) have found that curcumin reduces oxidative damage and amyloid pathology in an Alzheimer transgenic mouse model. It has also been found to have a protective effect against the toxicity of certain drugs (Venkatesan, 1998; Venkatesan and Chandrakasan, 1995; Venkatesan, 1998; Venkatesan et al., 2000; Venkatesan et al., 1997; Punithavathi et al., 2000) in rats.

The kinetics of pH-dependent curcumin degradation in aqueous solution has been examined and found to be complex (Tonnesen and Karlsen, 1985). In Phase II clinical trials, Cheng et al. (2001) investigated the pharmacokinetics and toxicology of curcumin in humans. Curcumin was administered in doses of from 500 mg/day to 12,000 mg/day for 3 months. There was no treatment-related toxicity up to 8,000 mg/day. The serum concentration of curcumin usually peaked at 1 to 2 hours after oral intake and gradually declined over the next 12 hours. The average peak serum concentration after taking 8,000 mg of curcumin was only 1.77±1.87 μM. Urinary excretion of curcumin was undetectable.

These clinical studies indicate that although curcumin is exceedingly useful in the treatment of a wide range of pathological conditions, its usefulness is limited by its rapid clearance from the body. Improved formulations of curcumin designed to increase its systemic bioavailability can improve the therapeutic potential of this valuable drug.

One method of administration of curcumin which has therapeutic potential is aerosol administration. An aerosol refers to a dispersion in air of solid or liquid particles, of fine enough particle size and consequent low settling velocities, to have relative airborne stability (Knight, 1973). Small particle aerosol treatment delivers a high dose of drug to the epithelium of the respiratory tract in amounts largely unachievable by other routes of administration (Knight, 1973). There is a subsequent steady rate of absorption of drug into the systemic circulation. Liposome aerosols consist of aqueous droplets within which are dispersed one or more particles of liposomes or liposomes containing one or more medications intended for delivery to the respiratory tract of a subject. Aerosol formulations of curcumin that incorporate lipids capable of forming liposomes would be beneficial in providing alternative means to deliver the drug systemically and improve its systemic bioavailability for the treatment of systemic disease. Aerosol-lipid formulations of curcumin would also provide a means to provide high doses of the drug to the respiratory epithelium for the treatment of pulmonary disease. Absorption of curcumin into the systemic circulation would be improved, thereby increasing the drug's bioavailability. These novel formulations of curcumin can be applied in the treatment of the vast range of pathological conditions which have been shown to be responsive to curcumin.

### BRIEF SUMMARY OF THE INVENTION

Accordingly, one of the objects of the present invention is to provide novel pharmaceutical lipid vehicle compositions of curcumin, one or more lipids, and an aqueous solvent. The inventor has created certain novel lipid vehicle compositions of curcumin and one or more lipids in an aqueous solvent that are suitable for aerosol delivery to a subject with beneficial properties. These compositions overcome the difficulty of fracture of liposomes following passage through a nebulizer. In addition, these compositions overcome the problem of limited bioavailability of curcumin following administration by other routes, such as orally and intravenously. As a result, these novel compositions can be applied in improved treatments of the many diseases in which curcumin has been implicated to have some benefit, such as cancer and asthma. The disease can be a systemic disease, such as a disorder associated with abnormal immune function, or a disease that is localized to a particular organ system such as the lungs.

Certain embodiments of the present invention include pharmaceutical lipid vehicle compositions suitable for aerosol delivery to a subject, the composition including curcumin, one or more lipids, and an aqueous solvent, wherein the transition temperature of the lipid, if only one lipid is present, or mean transition temperature of the lipids, if more than one lipid is present, is less than about 15° C., and the lipid vehicle composition can be nebulized. Curcumin, lipids, and aqueous solvents for use in the present invention are discussed further in other sections of this specification. Any concentration of curcumin for inclusion in the present pharmaceutical compositions is contemplated by the present invention. In certain embodiments, for example, the average aerosol concentration of curcumin is 0.115 mg/L of aerosol. Concentrations of curcumin and formulations involving curcumin are discussed elsewhere in this specification. Transition temperatures of lipids, aerosol formulations, and nebulization are discussed elsewhere in this specification.

In certain embodiments, the pharmaceutical compositions of the present invention include curcumin and one or more lipids dispersed as particles in an aqueous solvent. The particles include, but are not limited to, liposomes, lipid complexes, and any other particle that incorporates one or more lipids. The compositions can include a single lipid, or more than one lipid. Any type of lipid is contemplated for inclusion in the pharmaceutical compositions of the present invention, as long as the transition temperature of the lipid, if a single lipid, is less than about 15° C., or the mean transition temperature of the lipids, if more than one lipid is present, is less than about 15° C. One of ordinary skill in the art would be familiar with the wide range of lipids that are contemplated for inclusion in the present pharmaceutical compositions. Thus, for example, the lipid may be a phospholipid.

In certain embodiments of the present invention, the pharmaceutical composition includes liposomes. One of ordinary skill in the art would be very familiar with liposomes and methods to prepare liposomes. Liposomes are discussed in further detail elsewhere in this specification. Any method to prepare liposomes is contemplated by the present invention. Several examples of methods to prepare liposomes are discussed elsewhere in this specification.

In aspects of the present pharmaceutical compositions that include liposomes, it is contemplated that any number of the liposomes in the lipid vehicle composition can be nebulized without fracturing, as long as there is at least one liposome in the composition that can be nebulized without fracturing. Fracturing is defined to mean disruption of the liposome structure such that the structure no longer has the characteristics and properties of a liposome. One of ordinary skill in the art would be familiar with the characteristics and properties of liposomes, and methods to define whether these characteristics and properties exist. In certain embodiments of the present pharmaceutical compositions, a substantial portion of the liposomes can be nebulized without fracturing. A substantial portion is defined herein to mean more than about 25% of the liposomes. Thus, in certain more preferential embodiments of the present pharmaceutical compositions, greater than about 50% of the liposomes can pass through a nebulizer without fracturing. In certain even more preferential embodiments of the present compositions, greater than about 75% of the liposomes can pass through a nebulizer without fracturing. In certain more preferred embodiments, greater than 90% of the liposomes can pass through nebulizer without fracturing.

In some embodiments of the present invention, the liposomes form a complex with curcumin. Any type of complex is contemplated by the present invention. One of ordinary skill in the art would be familiar with drug-liposome complexes. In addition, any type of liposome is contemplated for inclusion in the pharmaceutical compositions of the present invention. For example, the liposomes may be unilamellar liposomes. In other embodiments, the liposomes are multilamellar liposomes. Any size and configuration of liposome is contemplated by the present invention. For example, in some embodiments, the liposomes have an average mass median aerodynamic diameter of about 1 to about 3 microns after being nebulized.

The present invention contemplates that any amount of curcumin in the lipid vehicle that is nebulized will be incorporated into the aerosol that is generated as a result of nebulization. In one embodiment, the average aerosol concentration of curcumin is 0.115 mg/L of aerosol.

In certain embodiments of the present invention, the lipid vehicle composition can be nebulized without excluding a substantial amount of the curcumin from the aerosol that is generated as a result of the nebulization of the lipid vehicle. A substantial amount of curcumin is defined herein to mean about 10% or less of the curcumin in the lipid vehicle composition. Thus, in certain embodiments of the present pharmaceutical composition, less than about 10% of the curcumin in the lipid vehicle is excluded from the aerosol. In certain more preferred embodiments, less than about 8% of the curcumin in the lipid vehicle is excluded from the aerosol. In certain even more preferred embodiments, less than about 6% of the curcumin in the lipid vehicle is excluded from the aerosol. In further even more preferred embodiments, less than about 4% of the curcumin in the lipid vehicle is excluded from the aerosol. In more preferred embodiments, less than about 2% of the curcumin in the lipid vehicle is excluded from the aerosol. In a most preferred embodiment, less than about 1% of the curcumin in the lipid vehicle is excluded from the aerosol that is generated following nebulization of the lipid vehicle.

As previously noted, any type of lipid or lipids is contemplated for inclusion in the pharmaceutical compositions of the present invention. Lipids are discussed in detail in other parts of this specification. The lipids that are contemplated for inclusion in the present pharmaceutical compositions may include any type of moiety incorporated into the lipid molecule. For example, in some embodiments, one or more of the lipids in the pharmaceutical composition include a fatty acid moiety that is fully saturated. In other embodiments, one or more of the lipids includes a 12-carbon fatty acid moiety. In further embodiments, one or more of the lipids includes two 12-carbon fatty acid moieties. In certain embodiments, the present pharmaceutical compositions include dilaurylphosphatidylcholine. In other embodiments, the pharmaceutical compositions of the present invention include one or more lipids that includes a monounsaturated fatty acid moiety or a polyunsaturated fatty acid moiety.

As previously noted, the lipid or lipids that are included in the pharmaceutical compositions of the present invention must have a transition temperature of less than about 15° C. if one lipid is present, or a mean transition temperature of less than about 15° C. if more than one lipid is present. In certain embodiments of the present invention, the transition temperature of the lipid or mean transition temperature of the lipids is less than about 5° C. In other embodiments, the transition temperature of the lipid or mean transition temperature of the lipids is less than about 0° C. In further embodiments, the transition temperature of the lipid or mean transition temperature of the lipids is greater than about −5° C.

In some embodiments, the pharmaceutical compositions of the present invention include one or more anti-hyperproliferative agents in addition to curcumin, one or more lipids as discussed above, and an aqueous solvent. The definition of anti-hyperproliferative agent includes any agent known to those of skill in the art that may be applied in the therapy of any disease characterized by proliferation of cells. For example, anti-hyperproliferative agents can be agents that are applied in the treatment of cancer. One of ordinary skill in the art would be familiar with this broad class of agents. Anti-hyperproliferative agents are discussed further in other parts of this specification.

In certain embodiments, the anti-hyperproliferative agents that are included in the pharmaceutical compositions of the present invention are chemotherapeutic agents. Inclusion of any chemotherapeutic agent known to those of ordinary skill in the art is contemplated for inclusion in the pharmaceutical compositions of the invention. For example, in certain embodiments the chemotherapeutic agents may include doxorubicin, daunorubicin, mitomycin, actinomycin D, bleomycin, cisplatin, VP16, an enedyine, taxol, vincristine, vinblastine, carmustine, melphalan, cyclophosphamide, chlorambucil, busulfan, lomustine, 5-fluorouracil, gemcitabin, BCNU, or camptothecin. The compositions may include a single chemotherapeutic agent, or more than one chemotherapeutic agent. Chemotherapeutic agents are discussed in more detail elsewhere in this specification.

Further embodiments of the present invention pertain to methods of treating a pathological condition in subject, including providing a lipid vehicle composition that includes curcumin, one or more lipids, and an aqueous solvent, wherein the transition temperature of the lipid or mean transition temperature of the lipids is less than about 15° C., and wherein the lipid vehicle composition can be nebulized, and administering the composition to the subject by inhalation. The methods of the present invention can be used in the treatment of any type of subject. In certain embodiments, the subject is a human subject. For example, the subject may be a cancer patient.

In some embodiments of the present methods, the subject inhales 5% CO₂ either before or during administration of the pharmaceutical composition to the subject. Administration of 5% CO₂, which is discussed further in other parts of this specification, facilitates inhalational administration of the composition since the 5% CO₂ promotes increased frequency and depth of respiration in a subject. More specifically, the 5% CO₂ is incorporated into a supply of air that flows through a nebulizer to generate the aerosol of the present invention. Still further, the aerosol generating gas may comprise 50% oxygen and 45% nitrogen and 5% CO₂. The increased oxygen percentage diminishes any feeling of air hunger which may occur with the inhalation of 5% CO₂.

As previously noted, the methods of the present invention include providing a lipid vehicle composition as previously defined and administering the composition to the subject. Any of the previously described lipid vehicle compositions of the present invention discussed above in this summary is contemplated for inclusion in the present methods of treatment. The detailed description of embodiments pertaining to the claimed compositions applies to the claimed methods. Thus, for example, certain embodiments of the claimed methods of the present invention involve use of compositions wherein the lipid vehicle includes liposomes, as discussed above in this summary.

The present invention pertains to methods of treating a pathological condition using the pharmaceutical compositions of the present invention. There is evidence that curcumin is of benefit in the treatment of a wide range of pathological conditions. These pathological conditions are discussed in detail elsewhere in this specification. The following list of conditions is by way of example. One of ordinary skill in the art would be familiar with the wide range of conditions that are amenable to treatment with curcumin.

In certain embodiments the pathological condition is a hyperproliferative disease. Hyperproliferative diseases are discussed above and elsewhere in this specification. For example, the hyperproliferative disease may be cancer. Any form of cancer is contemplated for treatment by the methods of the present invention. For example, the cancer may be breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, leukemia, or multiple myeloma.

In certain embodiments of the present invention, the method of treating a hyperproliferative disease in a subject further includes administering to the subject a therapeutically effective amount of a secondary anti-hyperproliferative agent. In certain embodiments the secondary anti-hyperproliferative agent is a secondary anti-cancer agent. There are numerous secondary anti-cancer agents that are available, and these forms of therapy are well-known to those of ordinary skill in the art. Examples of secondary anti-cancer therapies include, but are not limited to, chemotherapy, radiation therapy, surgery, immunotherapy, gene therapy, and hormonal therapy. These forms of therapy are discussed further in other sections of this specification.

In certain embodiments, the secondary anti-hyperproliferative agent is a chemotherapeutic agent. Any chemotherapeutic agent is contemplated for inclusion in the methods of the present invention. One of ordinary skill in the art would be familiar with the wide range of chemotherapeutic agents that are available. Examples of chemotherapeutic agents include doxorubicin, daunorubicin, mitomycin, actinomycin D, bleomycin, cisplatin, VP16, an enedyine, taxol, vincristine, vinblastine, carmustine, melphalan, cyclophosphamide, chlorambucil, busulfan, lomustine, 5-fluorouracil, gemcitabin, BCNU, or camptothecin.

In order to increase the effectiveness of the lipid vehicle compositions disclosed herein, it may be desirable to combine the lipid vehicle composition with the secondary anti-hyperproliferative agent. These compositions would be provided in a combined amount effective to provide for a therapeutic response in a subject. One of ordinary skill in the art would be able to determine whether the subject demonstrated a therapeutic response. This process may involve administering the lipid vehicle composition and the secondary anti-hyperproliferative agent to the subject at the same time. This may be achieved by administering a single composition or pharmacological formulation that includes both agents, or by administering two distinct compositions or formulations, at the same time, wherein one composition includes the lipid vehicle composition and the other includes the secondary agent.

Alternatively, the administration of the lipid vehicle composition may precede or follow the treatment with the secondary agent by intervals ranging from minutes to weeks. In embodiments where the secondary agent and lipid vehicle composition are separately administered, one would generally ensure that a significant period of time did not expire between the time of each delivery, such that the secondary agent and lipid vehicle composition would still be able to exert a beneficial effect on the subject. In such instances, it is contemplated that one may administer both modalities within about 24-48 h of each other and, more preferably, within about 12-24 h of each other, and even more preferably within about 30 minute-6 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

In other embodiments of the present invention, pathological condition is hypercholesterolemia. Pathological conditions affecting the skin are also contemplated by the present invention. For example, the methods of the present invention may be applied to promote wound healing and prevent skin wrinkling.

In other embodiments, the pathological condition is an inflammatory condition. Any inflammatory condition affecting any part of the body is contemplated by the present invention. In certain embodiments, the inflammatory condition is a pulmonary inflammatory condition, such as asthma.

The pathological condition may also be an arthritic condition. For example, in certain embodiments the pathological condition is rheumatoid arthritis. Alternatively, the pathological condition may be a viral infection, such as HIV infection. The pharmaceutical compositions of the present invention can also be applied, in certain embodiments, to the treatment of disease that is limited to the lungs. In further embodiments, the pathological condition to be treated is a systemic condition, such as a condition associated with abnormal immune function. For example, the pathological condition may be a cerebrodegenerative disease, such as multiple sclerosis. In further embodiments, the pathological condition to be treated is diabetes or heart disease, such as myocardial infarction or myocardial ischemia.

As used herein the specification, “a” or “an” may mean one or more. As used herein in the claim(s), when used in conjunction with the word “comprising”, the words “a” or “an” may mean one or more than one. As used herein “another” may mean at least a second or more.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Distribution of curcumin as a function of aerosol droplet size. Droplet size was measured with an Andersen cascade impactor. Aerosol was generated with an Aerotech II nebulizer flowing at 10 L of air/min. The initial starting concentration of curcumin contained in dilaurylphosphatidylcholine (DLPC) liposomes was 5.8 mg/mL. The concentration of drug to lipid was 1:10.

FIG. 2. Aerosol concentrations of curcumin during a 15-minute nebulization period. Initial curcumin concentration of the DLPC liposome preparation (1:10 ratio of drug to lipid) was approximately 6 mg/mL. Aerosol was generated with an Aerotech II nebulizer flowing at 10 L/min. Samples (2 min each) were collected from the nebulizer with all-glass impingers and drug analysed spectrophotometrically (UV, 420 nm).

### DETAILED DESCRIPTION OF THE INVENTION

Curcumin (diferuloylmethane) is an active component of turmeric (Curcuma longa), used as a spice and medicinal agent for centuries in India. Curcumin has a variety of therapeutic effects. For example, it has been shown to suppress carcinogenesis of the skin, liver, lung, colon, stomach, and breast. It has also been shown to lower blood cholesterol, promote wound healing, prevent skin wrinkling, inhibit inflammation, suppress rheumatoid arthritis, and inhibit HIV replication. Curcumin mediates this wide variety of therapeutic effect through the regulation of NF-κB and AP-1, suppression of IκBα kinase and c-jun N-terminal kinase, and inhibition of expression of cyclooxygenase 2, cyclin D1, adhesion molecules, matrix metalloproteinases, inducible nitric oxide synthase, HER2, EGF receptor, bcl-2, bcl-x1, and TNF. Curcumin has also been shown to inhibit the cell proliferation of a wide variety of tumor cells in culture and promote apoptosis through BID cleavage, cytochrome C release, and caspase-9 activation followed by caspase-3 activation. Pharmacologically, curcumin is quite safe, and doses as high as 8 g/day have been administered orally to humans with no side effects. Systemic bioavailability is very limited following oral administration.

The present invention is based on the inventors' discovery of novel pharmaceutical compositions of curcumin suitable for administration by inhalation. Studies of aerosolized lipid formulations of curcumin have been minimal. One study discussed non-lipid (dry) aerosol formulations of curcumin (U.S. Patent Application Publication 20030149113). Adequate systemic administration of dry aerosol formulations of curcumin are of limited benefit from a therapeutic standpoint, because the administration of dry aerosols for a period sufficient for systemic treatment would be intolerable to the patient. Moreover, lipid soluble substances may not be satisfactorily absorbed.

Other studies assessed non-aerosolized formulations of lipids and curcumin (U.S. Patent Application Publication 20020192274; U.S. Patent Application Publication 20010051184; U.S. Patent Application Publication 20010036919; U.S. Patent Application Publication 20010025034). These studies are limited by the low bioavailability that is currently associated with systemic (i.e., oral or parenteral) delivery of the liposome formulations. Enclosing curcumin in a liposome and dispersing it over the approximately 80 square meters of alveolar surface of the normal adult human lung provides a site for protected slow release of curcumin, locally in the lung or systemically through the approximately 70 square meters of blood capillaries that underlie the alveoli (see Weibel, 1963).

One study discussed the possibility of phospholipid compositions involving drugs for inhalation (U.S. Patent Application Publication 20020058009). However, no specific curcumin formulations were disclosed, and the inventors did not take into consideration the likelihood of fracturing and disruption of the drug-liposome complex following passage of the liposome through a nebulizer. Lipids used intravenously contain longer chain fatty acids that would not resist fracture when nebulized.

The inventors have discovered that pharmaceutical lipid vehicle compositions of curcumin suitable for aerosol delivery to a subject can be formulated using curcumin, one or more lipids, and an aqueous solvent, wherein the transition temperature of the lipid, if only one lipid is present, or mean transition temperature of the lipids, if more than one lipid is present, is less than about 15° C., and the lipid vehicle composition can be nebulized. In some embodiments, the composition includes liposomes. In certain embodiments, the pharmaceutical lipid vehicle composition includes dilaurylphosphatidylcholine (DLPC), which has a transition temperature of about 0° C.

The present invention also pertains to methods of treating pathological conditions using these novel pharmaceutical compositions. The pathological conditions include those disease wherein curcumin has been shown to be of benefit. In addition, aerosolized lipid compositions of curcumin may also be used to improve delivery of the drug to the respiratory epithelium, and can be used in the treatment of disease localized to the lung, such as asthma.

A. Curcumin

Commercial curcumin includes three major components: curcumin (77%), demethoxycurcumin (17%), and bisdemethoxycurcumin (3%), which are often referred to as “curcuminoids.” As used herein, “curcumin” is defined to include any one or more of these three major components of commercial curcumin, and any active derivative of these agents. This includes natural and synthetic derivatives of curcumin and curcuminoids, and includes any combination of more than one curcumenoid or derivative of curcumin. Derivatives of curcumin and curcumenoids include those derivatives disclosed in U.S. Patent Application Publication 20020019382, Kumar et al., 2000; Mishra et al., 2002; Dinkova-Kostova, 2002; Ohtsu et al., 2002; Ishida et al., 2002; Syu et al., 1998; Sugiyama et al., 1996; Osawa et al., 1995; Naito et al., 2002; Ruby et al., 1995; Rasmussen et al. 2000; Rao et al., 1984; Mukhopadhyay et al., 1982; Rao et al., 1982; Chun et al., 1999; Chun et al., 2002; and Kumar et al., 2003, each of which is herein specifically incorporated by reference.

Curcumin is insoluble in water and ether, but is soluble in ethanol, dimethylsulfoxide, and other organic solvents. It has a melting point of 183° C. and a molecular weight of 368.37. A detailed review of the properties and therapeutic potential of curcumin can be found in Aggarwal et al. (2003A), Aggarwal et al. (2003B), and Aggarwal et al. (2003C), each of which is herein specifically incorporated by reference for this section and all other sections of this application.

Any concentration of curcumin in the pharmaceutical lipid vehicle compositions of the present invention are contemplated. For example, in certain embodiments of the present invention, the concentration of curcumin in the pharmaceutical lipid vehicle (in mg/ml) is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 150, 200, 250, 300, 350, 400, 450, 500, and any concentration derivable therein or any range of concentrations derivable therein.

B. Lipids and Lipid Formulations

The present invention concerns pharmaceutical lipid vehicle compositions suitable for aerosol delivery to a subject that include curcumin, one or more lipids, and an aqueous solvent, wherein the transition temperature of the lipid, if only one lipid is present, or mean transition temperature of the lipids, if more than one lipid is present, is less than about 15° C., and the lipid vehicle composition can be nebulized.

As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

The lipids that are incorporated into the pharmaceutical lipid vehicle compositions of the present invention have a transition temperature (if a single lipid) or mean transition temperature (if more than one lipid) of less than about 15° C. The transition temperature is the point at which the phospholipid membranes become more fluid, changing from a tightly ordered “gel” or “solid” phase to a liquid-crystal phase where the freedom of movement of individual molecules is higher (see discussion in New, 1990). This parameter can be measured for a single lipid, or for a composition that includes more than one lipid. For example, the transition temperature of dilaurylphosphatidylcholine is about 0° C.

One example of a class of lipids includes the neutral fats. A neutral fat may comprise a glycerol and a fatty acid. A typical glycerol is a three carbon alcohol. A fatty acid generally is a molecule comprising a carbon chain with an acidic moiety (e.g., carboxylic acid) at an end of the chain. The carbon chain may of a fatty acid may be of any length, as long as lipid molecule has a transition temperature of less than about 15° C. or less. For example, the fatty acid may include from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. A fatty acid comprising only single bonds in its carbon chain is called saturated, while a fatty acid comprising at least one double bond in its chain is called unsaturated. Saturated and unsaturated lipids of the present invention are contemplated. For example, the lipid may contain fatty acid moieties that are fully saturated. Alternatively, in other embodiments, a fatty acid moiety in the lipid molecule may be monounsaturated or multiply unsaturated.

Another class of lipids includes phospholipids. A phospholipid generally comprises either glycerol or an sphingosine moiety, an ionic phosphate group to produce an amphipathic compound, and one or more fatty acids. Types of phospholipids include, for example, phosphoglycerides, wherein a phosphate group is linked to the first carbon of glycerol of a diglyceride, and sphingophospholipids (e.g., sphingomyelin), wherein a phosphate group is esterified to a sphingosine amino alcohol. For example, in some embodiments, the lipid molecule is dilaurylphosphatidylcholine, a phospholipid which includes two fully saturated fatty acid moieties.

A phospholipid may, of course, comprise further chemical groups, such as for example, an alcohol attached to the phosphate group. One of ordinary skill in the art would be familiar with the broad class of agents known as phospholipids, and the chemical groups which may be attached to phospholipids.

One of ordinary skill in the art would be familiar with the numerous other classes of lipids that are known to exist.

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources.

In the pharmaceutical compositions of the present invention, the curcumin is combined with one or more lipids and an aqueous solvent. Any concentration or amount of curcumin for dispersion in the lipid vehicle is contemplated by the present invention. For example, in certain embodiments the ratio of curcumin:lipid (wt:wt) may be 1:5, 1:6, 1:7, 1:8, 1:9, 1:10, 1:11, 1:12, 1:13, 1:14, 1:15, 1:20, 1:25, 1:30, 1:35, 1:40, 1:45, 1:50, or any ratio derivable therein.

One of ordinary skill in the art would be familiar with the range of techniques that can be employed for dispersing a drug in a lipid vehicle. For example, the curcumin may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes. However, the composition must be capable of forming a liposome that can pass through a nebulizer without fracturing. Liposomes and liposome formulations are discussed elsewhere in this specification.

The pharmaceutical compositions of the present invention involve curcumin dispersed in a lipid vehicle, and an aqueous solvent, such that the composition is suitable for aerosol delivery to a subject. Any aqueous solvent known to those of ordinary skill in the art is contemplated by the present invention. Any amount of aqueous solvent for inclusion in the present pharmaceutical compositions is contemplated for use in the present invention. Any method known to those of ordinary skill in the art can be used for combine the curcumin/lipid vehicle with the aqueous solvent. The lipid vehicle may be interspersed in the aqueous solution, possibly forming particles which are not uniform in either size or shape. In another example, they may be present in a bilayer structure, as micelles, or with a “collapsed” structure.

In certain embodiments, the lipid or lipids of the present pharmaceutical compositions may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of the present pharmaceutical compositions by weight or volume. Thus, it is contemplated that the lipid or lipids of the present pharmaceutical compositions may comprise any of the lipids, lipid types or other components in any combination or percentage range.

C. Aerosol Formulations

The present invention is directed to small particle aqueous aerosol particles containing lipids, lipid complexes, liposomes, and interacted curcumin-lipid or curcumin-liposome combination particles propelled or carried in air or oxygen-enriched air. The particles may also be propelled by carbon dioxide of about 5% in air supplied to the aerosol nebulizer to stimulate increased frequency and depth of breathing in animals and man (Koshkine et al., 2001), thereby to increase the residence time of inhaled particles in the respiratory tract, thus permitting a longer time for gravity, the major mechanism of deposition of small particles in the respiratory tract, to cause deposition of inhaled particles in the airways which will increase the administered dose without increased the amount of inhaled aerosol. If 5% carbon dioxide is used, oxygen-enriched air may be used instead of air. An increase in the oxygen percentage of the aerosol generating air results in a diminished feeling of air hunger which may occur with the inhalation of 5% carbon dioxide. U.S. Pat. Nos. 6,090,407, 4,427,649, 6,440,393, 6,375,980, 6,346,233, 5,958,378, 6,106,859, Knight et al. (1999), Knight et al., 1973, and Waldrep et al. (1994) contain information pertaining to formulations involving lipid, liposomes, and drug-liposome compositions for aerosol delivery, and each of these references is herein specifically incorporated by reference for this section and all other sections of this application.

An aerosol refers to a dispersion in air of solid or liquid particles, of fine enough particle size and consequent low settling velocities, to have relative airborne stability (Knight, 1973).

Lipid-drug combinations of the prior art are heterogeneous in size ranging from less than 1 micron up to 10 microns in diameter and have been given to patients in relatively large oral or intravenous doses. The outcome of such dosage is variable due to the differences in particle size of the preparation. Unexpectedly, the heterogeneous liposome particles and the interacted drug liposome combination particles can readily be converted to a more homogeneous small size by an aerosol nebulizer without any loss in effectiveness of the liposomes and interacted drug liposome combination particles while contained in aqueous aerosol particles of the above diameter size while propelled or carried in air or oxygen-enriched or carbon dioxide-enriched air. The concentration of the inhaled particles on the lung surface is also a unique advantage not afforded by the intravenous or other non-aerosol methods of administration.

Nebulizers are devices that use a compressor to aerosolize a medication, such as a liquid medication. They are widely used in the U.S. to delivery medications by inhalation. Any nebulizer known to those of skill in the art is contemplated for use in the present invention. One of ordinary skill in the art would be familiar with the use and characteristics of nebulizers.

Advantageously, these small particle aqueous aerosol droplets containing these lipid-, lipid complex- and liposome-drug combinations, when inhaled, provide high concentration on the respiratory epithelium and a steady rate of absorption into the circulation without the hazard of peak levels that may be associated with large oral or intravenous doses of drug, and deliver a high dose of drug to the epithelium of the respiratory tract in amounts largely unachievable by other routes of administration.

One to several lipid-, lipid complex-, or liposome-curcumin particles (<1 micron in diameter) may be contained in a single aerosol droplet (1-3 micron, aerodynamic mass median diameter) depending on the concentration of lipid material in the preparation that is to be nebulized. As previously mentioned, the advantage of such a discretely sized population of particles is that because of their small size and low settling velocities they will penetrate when inhaled into the lower respiratory tract in substantial percentages. For example, 1.5 micron particles will deposit 46% of the total inhaled in the lung and another 36% in the nose and upper air passages. Such uniform deposition permits treatment of lesions at any level of the respiratory tract and also provides an interface into the cell without the problems and disadvantages associated with oral and intravenous injections. Lipid formulations of drugs for aerosol administration are discussed in greater detail in U.S. Pat. No. 5,049,388, specifically incorporated by reference herein.

When deposition is desired in the alveolated portion of the lung, which occupies about 97% of the lung volume, the small particle aerosol is administered by mouth-only breathing. With this methodology, the total deposition at 1.6 μm mass median aerodynamic diameter (MMAD) is about 28%, of which about 21% will reach alveolated lung and the remainder will deposit in the mouth or the bronchial passages. Under some circumstances, such as primarily lung diseases include primary and metastatic lung cancer, medication is only desired in the lung, such as provided by mouth-only breathing.

Aerosol deposited in the nasopharynx, mouth, and upper airways is moved by muco-ciliary action to the esophageal orifice and swallowed over the course of an hour or so after inhalation, and thus is oral dosage. The unique feature of aerosol treatment is the deposition in the alveolated lung (i.e., lung periphery) adjacent to the large blood capillary network, whereby the drug may locally be deposited in tumors and other diseases or be absorbed into capillaries to be carried to the left side of the heart and distributed through the aorta and other arteries to organs and peripheral tissue by first pass (i.e, directly to the site of action, instead of intravenous administration, which is widely distributed and diluted by intravenous passage, which in turn diminishes the dose to a particular site) resulting in effective treatment.

Interaction of the drug or compound with lipids can be achieved in one or more ways such as 1) intercalated into the lipid bilayer, 2) covalently attached to a lipophilic compound (e.g., phospholipid) which is inserted into the lipid bilayer, 3) trapped between the phospholipid layers which comprise the lipid bilayer, or 4) a component soluble in the aqueous phase, but whose chemical properties (e.g., hydrophobic ionic, or Van der Waals forces) allow an interaction with the phospholipid bilayer such that when the liposome permeability barrier is damaged, the compound is not released from the liposome.

D. Liposome Preparations

Certain embodiments of the present invention pertain to liposome preparations. Selected lipids are dissolved in a lipid solvent such as tertiary butanol and on specific occasions with a small amount of dimethylsulfoxide (DMSO), refrigerated, and lyophilized under negative pressure. The dried material, when placed into an aqueous environment will spontaneously associate into multilamellar structures that function as permeability barriers. These lipid vesicles, termed liposomes, are composed of aqueous compartments separated from each other and the external medium by a series of closed concentric lipid bilayers.

The liposomes preferably are multilamellar, although unilamellar liposomes are also contemplated by the present invention, and the liposomes and interacted liposome-drug combinations may be prepared in any suitable manner known to those of ordinary skill in the art. Methods for preparation of liposomes are discussed in U.S. Pat. No. 4,370,349, and in Waldrep et al., 1994, which is specifically incorporated by reference herein.

The composition of the aqueous compartments is the same as the medium in which the liposomes were formed; this makes it possible to entrap a wide variety of materials within the lipid bilayers. Entrapped markers can be released by a variety of lytic agents in a manner analogous to natural membranes. Since liposomes may be prepared from substances found in normal cell membranes, they are perceived as nontoxic to mammalian host; and studies in humans and laboratory animals have supported this concept.

The ability to encapsulate water soluble compounds in liposomes led to their use as clinically as carriers of drugs. Studies with water insoluble anticancer and antimicrobial compounds have suggested that liposomes may be ideally suited for delivery of this type of drug. The amounts of drug associated with liposomes are high and release does not occur until the membranes are destroyed either by mechanical means of biodegradation, thus allowing a more controlled release of the drug over time. Moreover, in laboratory animals the use of liposomes actually reduced toxic effects observed with the drug alone.

Procedures for preparation of liposomes have in common the dispersal of a phospholipid or mixture of lipids into a suitable container and the removal of an organic solvent, for example, ether, chloroform, or T-butanol, by methods such as evaporation, rotary evaporation under vacuum or lyophilization with commercially available freeze-drying equipment. Dispersing the resulting lipid film of dry lipid powder in an aqueous medium, for example, distilled water, isotonic saline or buffered solutions will result in the formation of liposomes. For example, phosphatidylcholine is dissolved in re-distilled T-butanol and transferred to a bottle. The solution is frozen and the solvent is removed under vacuum using a commercial freeze-dryer. Sterile pyrogen-free distilled water is added to the freeze dried powder and the bottle shaken to disperse the powder. The resulting milky suspension can be examined microscopically and the suspension shown to contain liposomes that are heterogeneous in size ranging from less than 1 micron up to 10 microns.

Curcumin may be associated with the lipid formulations either as the sole drug in the composition, or as part of a group of more than one drug. For example, some embodiments of the present invention involve incorporation of anti-hyperproliferative agents in the pharmaceutical lipid-curcumin compositions of the present invention. Anti-hyperproliferative agents are discussed elsewhere in this specification. Any other drug known to those of ordinary skill in the art can be included in the curcumin compositions of the present invention. Whether the curcumin is associated with the lipid portion of the liposomes or resides in the aqueous compartments is dependent upon the physical and chemical properties of the compound of biological interest. It is understood that the procedures used for preparing liposome-drug combinations are not restricted under this invention, any procedure that results in liposomes would be applicable. For purposes of disclosure, two general methods of producing interacted drug liposomes are described below. They illustrate that regardless of chemical and physical properties a wide array of biologically active compounds or medications can be interacted with liposomes and that such liposomes are applicable to delivery by small particle aerosol.

Method I may be used to incorporate lipid soluble or lipid-bound biologically active compounds or medications. For example, a suitable lipid dissolved in an organic solvent is transferred to a suitable flask or bottle. The curcumin is added, the solution is frozen and the solvent is removed using a commercial freeze-dryer. Liposomes are formed by the addition of a suitable aqueous medium, for example, sterile distilled water, isotonic saline or a buffered solution followed by vigorous shaking of the container. It is recognized that the phospholipid or mixture of phospholipids used to prepare the liposomes can be altered to increase or decrease the lipid solubility of the active compound as desired and that solvents such as chloroform, n-butanol, t-butanol, or pyridine may be used to promote interaction of the compound and phospholipid. The specific procedure can be tailored to accommodate the individual properties of specific compounds.

Method II may be used to incorporate biologically active compounds or medications without regard to their solubility characteristics. In this procedure the curcumin is covalently attached to a lipid with the result that the lipid moiety will associate with the liposome and anchor the compound to the liposomal biolayers. In this procedure the compound and a lipid derivative capable of derivatizing the compound are mixed in a suitable solvent and allowed to react. The lipid derivative of the compound is then purified and incorporated into liposomes. For example, the lipid and appropriate quantities of lipid derivatized curcumin are dissolved in an organic solvent and added to a suitable flask or bottle. The solution is frozen and the solvent removed in a freeze-dry apparatus. Liposomes are then formed by addition of suitable aqueous medium to the dry powder, followed by vigorous shaking of the container. It is recognized that a wide variety of chemical reactions can be utilized to prepare lipid derivatives of biologically active compounds and that alternative procedures will be suitable, if the resulting derivative can be interacted with liposomes and if the biological activity of the curcumin has not been inactivated by the process. It is also recognized that lipid derivatives of some compounds, for example peptides, proteins or hormones may be efficiently incorporated when added in the aqueous medium rather than to the organic solvent.

The protein 11-2, for example, may be incorporated into a liposome using dimyristoylphosphatidylcholine (DMPC) diluted in saline containing sodium phosphate buffer at pH 7 and 25 mg of human serum albumin/ml. One ml of the aqueous 11-2 can be added to 300 mg of DMPC powder contained in a 50-ml plastic centrifuge tube and the resulting milky appearing solution subjected to 3 cycles of vortex mixing, bath sonication, freezing in a dry ice/ethanol bath and thawing. The resulting solution can then be diluted 7.5 times in phosphate buffered saline to achieve a final concentration of 2×10⁶ U Il-2/ml, 40 mg DMPC/ml and 3.3 mg HSA/ml. 11-2 liposome material (0.5 ml per unit dose vial) can then be aliquoted into 5 ml glass serum vials which can be stoppered, capped and stored at 4° C. A detailed description of this method of preparation of liposomes can be found in Anderson et al. (1992), which is hereby specifically incorporated by reference.

E. Pharmaceutical Compositions and Routes of Administration

1. Overview

An objective of the present invention was to develop aerosol formulations of curcumin to achieve improved bioavailabilities as well as improved delivery of curcumin to the respiratory epithelium. The invention stems from the desire to overcome the problem of limited and erratic oral bioavailability following oral administration and administration by other routes.

2. Effective Amount

Pharmaceutical compositions of the present invention will include an effective amount of a curcumin and additional therapeutic agent, if one is present, that is clinically determined to be useful in the treatment of the particular disease under consideration. One of ordinary skill in the art would be familiar with what type of dosage is required for treatment of the particular pathological condition that is present in the subject. No undue experimentation would be involved. When used for therapy, the compositions of the present invention are administered to subjects in therapeutically effective amounts. For example, an effective amount of curcumin in a patient with cancer may be an amount that promotes tumor regression. Alternatively, an effective amount of curcumin may be an amount that promotes resolution of pulmonary inflammation. The dose will depend on the nature of the disease, the subject, the subject's history, and other factors. Preparation of such compositions is discussed in other parts of this specification. In some embodiments of the present invention, for example, a 1-hour treatment at 10 mg curcumin/ml would deposit an estimated 0.186 mg of curcumin/kg of body weight in a subject. In some embodiments of the present invention, 5% CO₂ will be administered to the subject prior to and/or during administration of the claimed composition. Carbon dioxide enhancement of inhalation therapy has been described in U.S. Pat. No. 6,440,393, which is herein specifically incorporated by reference.

In cancer patients, the access to aerosolized curcumin will be particularly important, since their intestinal absorption is often perturbed after chemotherapy, aggravating the already erratic intestinal absorption of various medications. Further, the availability of curcumin for more effective systemic administration will make it possible to clinically compare the effect of higher systemic doses of curcumin to other medications as well as to lower doses of curcumin.

3. Definition of “Pharmaceutical”

The phrases “pharmaceutical,” “pharmaceutically,” or “pharmacologically acceptable” refer to molecular entities and compositions that do not produce an unacceptably adverse, allergic or other untoward reaction when administered to an animal, or human, as appropriate. As used herein, “pharmaceutical” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients to treat the disease of interest, such as other anti-cancer agents or anti-inflammatory agents, can also be incorporated into the compositions.

4. Compositions Suitable for Aerosol Administration

As discussed in the summary of the invention, curcumin will be formulated for inhalational administration. Any method of preparation of the compositions for inhalational administration is contemplated for use in administering the pharmaceutical compositions in the claimed methods. One of ordinary skill in the art would be familiar with these methods of administration of a pharmaceutical composition. In certain embodiments, a nebulizer is used to generate an aerosol for administration to the subject. One of ordinary skill would be familiar with use and characteristics of nebulizers.

The definition of aerosol is discussed elsewhere in this specification. Typically, the compositions can be prepared as liquid solutions or suspensions for aerosol administration using a nebulizer; solid forms suitable for using to prepare solutions or suspensions upon the addition of a liquid prior to aerosol administration can also be prepared.

5. Formulation Principles

Solutions of curcumin dispersed in a lipid will be prepared in an aqueous solvent. These preparations may contain a preservative to prevent the growth of microorganisms.

The pharmaceutical preparations will be sterile. It must be chemically and physically stable under the conditions of manufacture and storage and must be preserved against the contaminating action of microorganisms, such as bacteria and fungi.

The carrier also can any aqueous solvent known to those of ordinary skill in the art. The prevention of the action of microorganisms can be brought about by various antibacterial and antifungal agents, for example, parabens, chlorobutanol, phenol, sorbic acid, thimerosal, and the like. In many cases, it will be preferable to. include isotonic agents, for example, sugars or sodium chloride.

Sterile injectable solutions are prepared by incorporating the active compounds in the required amount in the appropriate solvent with various of the other ingredients enumerated above, as required, followed by filtered sterilization.

Upon formulation, solutions will be administered in a manner compatible with the dosage formulation and in such amount as is therapeutically effective. One of ordinary skill in the art would be familiar with techniques for administration of an aerosol.

6. Methods of Measuring Concentration of Drugs in a Composition

Following preparation of the pharmaceutical compositions of the present invention, it may be desirable to quantify the amount of curcumin in the pharmaceutical composition. Methods of measuring concentration of a drug in a composition include numerous techniques that are well-known to those of skill in the art. Selected examples include chromatographic techniques. There are many kinds of chromatography which may be used in the present invention: drug-specific assays, adsorption, partition, ion-exchange and molecular sieve, and many specialized techniques for using them including column, paper, thin-layer chromatography, gas chromatography, and high performance liquid chromatography (HPLC). One of ordinary skill in the art would be familiar with these and other related techniques.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

7. Pathological Conditions to be Treated

As noted in other parts of this specification, there is substantial evidence that curcumin would be beneficial in the treatment of a wide variety of pathological conditions. These conditions are specifically detailed in Aggarwal et al. (2003A), Aggarwal et al. (2003B), and Aggarwal et al. (2003C), which have been specifically incorporated by reference in the section pertaining to curcumin, and are specifically incorporated by reference for purposes of this section.

Examples of pathological conditions responsive to curcumin therapy include, but are not limited to, hyperproliferative diseases such as cancer. Curcumin inhibits proliferation of breast carcinoma cell lines in culture (Mehta et al., 1997; Ramachandran and You, 1999; Simon et al., 1998). HER2/neu and EGFR activity represent one possible mechanism by which it suppresses the growth of breast cancer cells. It has been shown to down regulate the activity of EGFR (Korutla et al., 1994; Korutla et al., 1995) and HER2/neu (Korutla et al., 1994; Korutla et al., 1995) and to deplete the HER2/neu protein (Hong et al., 1999). A variety of other mechanisms against cancer are proposed, and are discussed elsewhere in this specification.

There is evidence that curcumin is beneficial in the treatment of atherosclerosis and myocardial infarction. It is also implicated to be beneficial in suppressing diabetes. Curcumin has also been shown to stimulate muscle regeneration and enhance wound healing. Curcumin also suppresses the symptoms associated with arthritis, such as rheumatoid arthritis. It has also been shown to reduce the incidence of gall-stone formation. Curcumin modulates multiple sclerosis and cerebrodegenerative diseases. It also blocks the replication of HIV. Curcumin has also been shown to have a beneficial effect on Alzheimer disease. In addition, it protects against cataract formation in lenses. Curcumin also protects from drug-induced toxicity, such as drug-induced myocardial toxicity, drug-induced lung injury, and drug-induced nephrotoxicity. There is also evidence that curcumin is beneficial in the treatment of skin wrinkling, and inflammatory diseases such as inflammatory lung disease and asthma. One of ordinary skill in the art would be familiar with the wide range of diseases conditions for which curcumin has been implicated and shown to be beneficial, and each of these diseases is contemplated for treatment by the pharmaceutical compositions of the present invention.

F. Secondary Anti-Hyperproliferative Therapies

1. General

Some embodiments of the claimed methods of the present invention involve administering to the subject a therapeutically effective amount of a secondary anti-hyperproliferative therapy. In certain embodiments, the secondary anti-hyperproliferative therapy is an anti-cancer therapy. More particularly, in certain embodiments, the anti-cancer therapy is chemotherapy.

A wide variety of cancer therapies, known to one of skill in the art, may be used in combination with the compositions of the claimed invention. Some of the existing cancer therapies and chemotherapeutic agents are described below. One of skill in the art will recognize the presence and development of other anticancer therapies which can be used in conjugation with the lipid vehicle compositions and will further recognize that the use of the secondary therapy will not be restricted to the agents described below.

In order to increase the effectiveness of the lipid vehicle compositions disclosed herein, it may be desirable to combine the lipid vehicle composition with the secondary anti-hyperproliferative agent. These compositions would be provided in a combined amount effective to provide for a therapeutic response in a subject. One of ordinary skill in the art would be able to determine whether the subject demonstrated a therapeutic response. This process may involve administering the lipid vehicle composition and the secondary anti-hyperproliferative agent to the subject at the same time. This may be achieved by administering a single composition or pharmacological formulation that includes both agents, or by administering two distinct compositions or formulations, at the same time, wherein one composition includes the lipid vehicle composition and the other includes the secondary agent. Still further, the present invention may also include the use multiple secondary anti-cancer agents or therapies, for example, gene therapy in combination with chemotherapy and radiation. In certain embodiments, gene therapy may be administered by aerosol.

Alternatively, the administration of the lipid vehicle composition may precede or follow the treatment with the secondary agent by intervals ranging from minutes to weeks. In embodiments where the secondary agent and lipid vehicle composition are separately administered, one would generally ensure that a significant period of time did not expire between the time of each delivery, such that the secondary agent and lipid vehicle composition would still be able to exert a beneficial effect on the subject. In such instances, it is contemplated that one may administer both modalities within about 24-48 h of each other and, more preferably, within about 12-24 h of each other, and even more preferably within about 30 minute-6 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, the lipid vehicle composition is “A” and the secondary agent, such as chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

Administration of the lipid vehicle compositions of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

2. Radiotherapy

Radiotherapy include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In the context of the present invention radiotherapy may be used in addition to the lipid vehicle compositions of the invention to achieve cell-specific cancer therapy.

3. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, in the context of the present invention surgery may be used in addition to treatment with the lipid vehicle composition.

In the case of surgical intervention, the compositions of the present invention may be used preoperatively, to render an inoperable tumor subject to resection. Alternatively, the present invention may be used at the time of surgery, and/or thereafter, to treat residual or metastatic disease.

In certain embodiments, the tumor being treated may not, at least initially, be resectable. Treatments with the lipid vehicle compositions of the present invention may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

4. Chemotherapeutic Agents

Certain embodiments of the present pharmaceutical compositions include pharmaceutical compositions of curcumin suitable for aerosol delivery wherein the pharmaceutical composition includes one or more anti-hyperproliferative agents. In certain embodiments, the anti-hyperproliferative agents are chemotherapeutic agents that are used in cancer therapy. Any chemotherapeutic agent is contemplated for inclusion in the compositions and methods of treatment of the present invention, and the wide range of chemotherapeutic agents that are available are well-known to those of skill in the art. Examples of these agents include cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin, BCNU, gemictabin, camptothecin, and methotrexate or any analog or derivative variant thereof. The term “chemotherapy” as used herein is defined as use of a drug, toxin, compound, composition or biological entity which is used as treatment for cancer. These can be, for example, agents that directly cross-link DNA, agents that intercalate into DNA, and agents that lead to chromosomal and mitotic aberrations by affecting nucleic acid synthesis.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like.

Other anti-hyperproliferative agents contemplated by the present invention include immunotherapeutics. Immunotherapeutics generally rely on the use of immune effector cells and molecules to target and destroy cancer cells. Other types of anti-hyperproliferative agents that are contemplated for use in the compositions of the present invention include agents used in toxin therapy and hormonal therapy. One of skill in the art would know that this list is not exhaustive of the types of agents that are available as anti-hyperproliferative agents.

5. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with the claimed lipid vehicle compositions of the present invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

6. Genes

In yet another embodiment, the secondary treatment is a gene therapy in which a non-p53 expression cassette is administered before, after, or at the same time as a p53 expression cassette. Delivery may comprise use of a vector encoding p53 in conjunction with a second vector encoding an additional gene product. Alternatively, a single vector encoding both genes may be used. Still further, gene therapy may be administered via an aerosol. A variety of secondary gene therapy proteins are envisioned within the invention, some of which are described below.

7. Other Cancer Therapies

Examples of other cancer therapies include phototherapy, cryotherapy, toxin therapy, or hormonal therapy. One of skill in the art would know that this list is not exhaustive of the types of treatment modalities available for cancer and other hyperplastic lesions.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials and Methods

Preparation of a pharmaceutical composition that includes curcumin, DLPC, and an aqueous solvent was based on the method described in Waldrep et al., 1994. This method was used to generate drug-liposomes.

Aerosol was generated with an Aerotech II nebulizer flowing at 10 L of air/min. Aerosol droplet size was measured with an Andersen cascade impactor. The initial starting concentration of curcumin contained in dilaurylphosphatidylcholine (DLPC) liposomes was 5.8 mg/mL. The concentration of drug to lipid was 1:10.

Aerosol concentrations of curcumin during a 15-minute nebulization period was measured in two independent experiments. Aerosol was generated with an Aerotech II nebulizer flowing at 10 L/min. Samples (2 min each) were collected from the nebulizer with all-glass impingers and drug analysed spectrophotometrically (UV, 420 nm).

### Example 2

Formulation and Aerosolization of Dilauroylphosphatidylcholine (DLPC)-Curcumin Liposomes

To optimize the formulation of dilaurylphosphatidylcholine (DLPC) and curcumin, drug (1 mg/mL) and various ratios of lipid, 1:2, 1:3, 1:5, and 1:10 (wt:wt), were prepared, lyophilized to dryness and resuspended in water were evaluated before and after aerosolization for liposome formation and stability of the drug-lipid complex. Polarized light microscopy revealed some crystal aggregates at ratios 1:5 and less. Next, the production of liposomes was scaled up so that formulations of 10 mg of curcumin/mL and 100 mg of DLPC/mL could be prepared. These preparations consisted of taking 0.1 mL of a stock solution of curcumin (500 mg/mL of DMSO) and adding it to 10 mL of t-butanol containing 500 mg of DLPC. The solution was frozen at −80° C. for 2 hr and lyophilized to dryness over a 24 hr period of time. The lyophilized powder was stored in the dark.

For nebulization, lyophilized curcumin-DLPC was resuspended in 5 mL of water (final concentration, 10 mg of curcumin/mL and 100 mg of DLPC/mL). The solution was added to the reservoir of an Aerotech II nebulizer. Compressed air flowing at 10 L/min generated the aerosol. Aerosol samples (2 min each) were taken at the beginning (1-3 min), middle (7-9) min) and end 13-15 min) of the aerosolization period. Aerosol was collected in 20 mL of water in an all-glass impinger (AGI) and the concentration of curcumin was determined by UV absorption at 420 nm and compared to a standard curve. Over the 15 min period of aerosolization, the average aerosol concentration of curcumin was 0.115 mg/L of aerosol. Table 1 demonstrates the effect of curcumin to lipid (DLPC) ratio on the generation of a liposome aerosol. The data demonstrate that a curcumin to lipid ratio of 1:10 makes a better formulation and produces an aerosol with greater curcumin content than a 1:3 formulation).

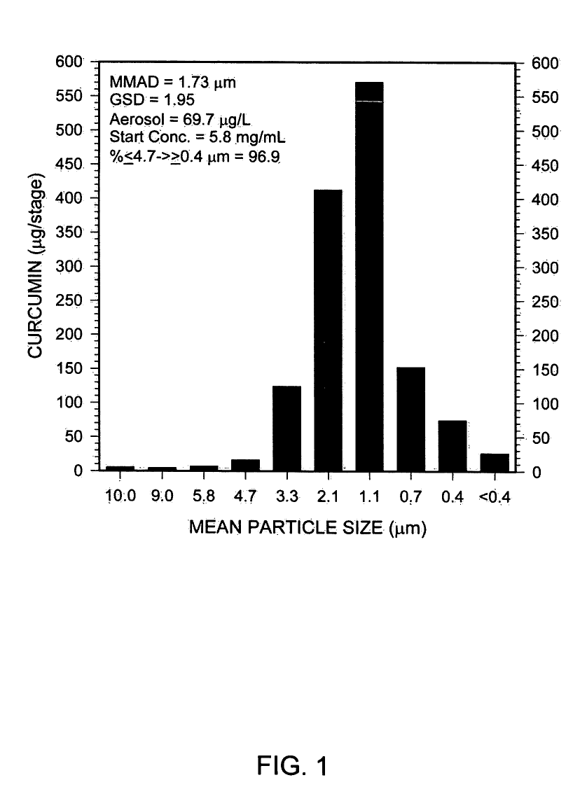

The distribution of curcumin as a function of aerosol droplet size is shown in FIG. 1. The distribution of droplet sizes with a mass median aerodynamic diameter (MMAD) of 1.73 μm and a geometric standard deviation (GSD) of 1.95 are typical of previous liposome aerosols generated with the Aerotech II nebulizer and are ideal for deposition of drug throughout the respiratory tract. The droplet distribution pattern indicates that 96.9 percent of the aerosol generated with the Aerotech II nebulizer will be within the “breathable range” (<4.7 to >0.4 μm). Aerosols with these characteristics are well suited for therapeutic uses.

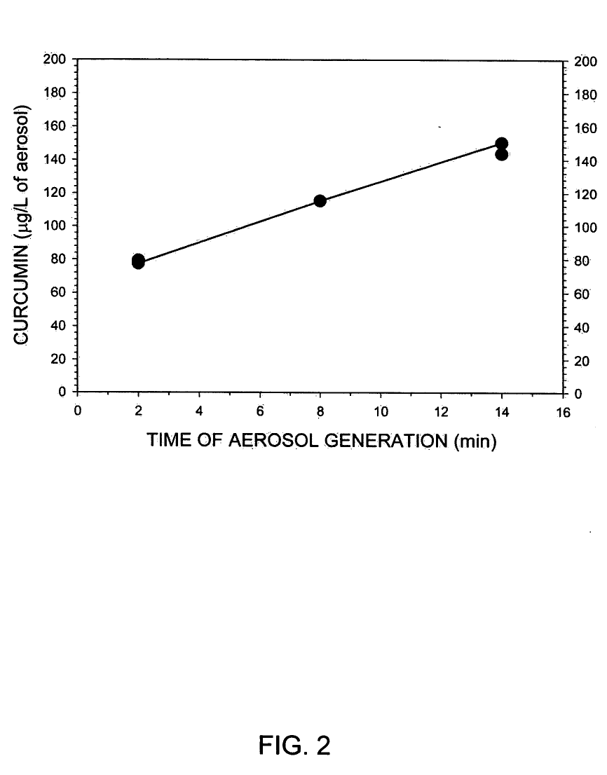

Aerosol concentrations of curcumin during a 15-minute nebulization period are shown in FIG. 2. Data represents two independent experiments. Initial curcumin concentration of the DLPC liposome preparation (1:10 ratio of drug to lipid) was approximately 6 mg/mL. Sampling of aerosols generated from an Aerotech II nebulizer containing curcumin-DLPC liposomes demonstrates a continuous production of curcumin in the aerosol over the period of time studied. The observed increase in curcumin aerosol concentration from the start to the end of the experiment is typical of jet-type nebulizers (e.g., Aerotech II) and is caused by excessive evaporation of water compared to the curcumin. A similar concentration effect was seen in the reservoir material (see Table I). These studies indicate that it will be possible to produce curcumin aerosols for periods of a few minutes to several hours for therapeutic purpose.

TABLE I
Effect of Curcumin to Lipid (DLPC) Ratio
on the Generation of a Liposome Aerosol

| | | | |
|---|---|---|---|
| Time of | Concentration of | Concentration of | Efficiency of |
| Sample | Curcumin in | Curcumin in | Aerosol |
| (min) | Reservoir (mg/mL) | Aerosol (μg/L) | Generatin (%) |

Ratio of Curcumin to DLPC was 1:3 by weight

| | | | |
|---|---|---|---|
| 0 | 11 | | |
| 1 | | 17.6 | 0.16 |
| 7 | | 67.4 | |
| 13 | | 114.8 | 0.27 |
| 14 | 42 | | |
| Mean | | 66.6 | |

Ratio of curcumin to DLPC was 1:10 by weight

| | | | |
|---|---|---|---|
| 0 | 6.6 | | |
| 1 | | 79.3 | 1.21 |
| 7 | | 115.2 | |
| 13 | | 150.3 | 1.00 |
| 11 | 15.1 | | |
| Mean | | 114.9 | |

In summary, curcumin can be formulated with DLPC to produce stable liposomes suitable for aerosol administration.

### Example 3

### Use of Aerosoled Curcumin Liposomes to Treat Hyperproliferative Diseases

The aerosol curcumin liposomes may be used in the treatment of hyperproliferative disease, such as cancer.

A curcumin liposome aerosol is administered using similar techniques as those described in Knight et al., 1999 and Verschraegen 2004 (each of which is incorporated by reference), such procedures may include daily administration of the aerosol treatment, five days per week. Treatment periods may vary from 15 to 120 minutes per day. Parameters relating to tumor growth, tumor size and survival are measured.

Still further, the present invention may be used in combination with other anticancer chemotherapies that have been aerosolized (Koshkina et al., 2001). It is envisioned that the combination will enhance the effect of curcumin.

### Example 4

### Use of Aerosoled Curcumin Liposomes to Enhance Anti-Cancer Therapies

It has recently been reported that curcumin induces caspase-3-independent apoptosis in human multidrug resistant cells (Piwocka et al., 2002). Multi-drug resistance develops principally due to overexpression of P-glycoprotein (P-gp). P-gp binds to structurally unrelated compounds and transports them out of the cell. Curcumin appears to act by bypassing multi drug resistance (mdr), thus the present invention is an alternative route to avoid drug resistance. Such techniques to determine the interaction of curcumin with P-gp are designed similar to those in which aerosolized cyclosporin A binds to P-gp and blocks the diffusion of paclitaxel out of the cell, thus increasing the anticancer effect of paclitaxel (Koshkina et al., 2004). Thus, it is envisioned that curcumin binds to P-gp and blocks the diffusion of a chemotherapeutic agent out of the cell, such as paclitaxel, resulting in an improvement in the anticancer effect of the chemotherapeutic agent.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

The following references, to the extent that they provide exemplary procedural or other details supplementary to those set forth herein, are specifically incorporated herein by reference.
U.S. Pat. No. 4,370,349
U.S. Pat. No. 4,427,649
U.S. Pat. No. 5,049,388
U.S. Pat. No. 5,958,378
U.S. Pat. No. 6,090,407
U.S. Pat. No. 6,106,859
U.S. Pat. No. 6,346,233
U.S. Pat. No. 6,375,980
U.S. Pat. No. 6,440,393
U.S. Patent Application Publication 20010025034
U.S. Patent Application Publication 20010036919
U.S. Patent Application Publication 20010051184
U.S. Patent Application Publication 20020058009
U.S. Patent Application Publication 20020192274
U.S. Patent Application Publication 20030149113
Aggarwal et al., Anticancer Res., 23(1A):363-398, 2003b.
Aggarwal et al., In: Herbal and Traditional Medicine: Molecular Basis of Health, Packer et al. (Eds.), M.D. Anderson Cancer Center, Texas, 2003a.
Aggarwal et al., In: Phytochemicals in Cancer Chemoprevention, Bagchi et al. (Eds.), M.D. Anderson Cancer Center, Texas, 2003c.
Ammon and Wahl, Planta Med., 57:1-7, 1991.
Anderson et al., J. of Immunotherapy, 12:19-31, 1992.
Arbiser et al., Mol. Med., 4:376-383, 1998.
Arun and Nalini, Plant Foods Hum. Nutr. 57:41-52, 2002.
Asai and Miyazawa, J. Nutr., 131:2932-2935, 2001.
Bharti et al., Blood, 101(3):1053-1062, 2003.
Chen and Huang, Br. J. Pharmacol., 124:1029-1040, 1998.
Chen and Tan, Oncogene, 17:173-178, 1998.
Chen et al., Anticancer Res., 19:3675-3680, 1999.
Cheng et al., Anticancer Res., 21:2895-2900, 2001.
Chuang et al., Carcinogenesis, 21:331-335, 2000.
Chun et al., J. Environ. Pathol. Toxicol. Oncol., 21(2):131-139, 2002.
Chun et al., Mutat. Res., 428(1-2):49-57, 1999.
Conney et al., Adv. Enzyme Regul., 31:385-396, 1991.
Deodhar et al., Indian J. Med. Res., 71:632-634, 1980.
Dikshit et al., Indian J. Med. Res., 101:31-35, 1995.
Dinkova-Kostova, Mini Rev. Med. Chem., 2(6):595-610, 2002.
Han et al., Clin. Immunol., 93:152-161, 1999.
Hong et al., Clin. Cancer Res., 5:1884-1891, 1999.
Huang et al., Cancer Res., 51:813-819, 1991.
Huang et al., Cancer Res., 54:5841-5847, 1994.
Huanget al., Carcinogenesis, 18:83-88, 1997.
Huang et al., Eur. J. Pharmacol., 221:381-384, 1992.
Hussain and Chandrasekhara, Indian J. Biochem. Biophys., 31:407-412, 1994.
Hussain and Chandrasekhara, Indian J. Med. Res., 96:288-291, 1992.
Ishida et al., Bioorg. Med. Chem., 10(11):3481-3487, 1998.
Kawamori et al., Cancer Res., 59:597-601, 1999.
Kim et al., Carcinogenesis, 19:81-85, 1998.
Knight et al., Cancer Chemother. Pharmacol., 44(3):177-186, 1999.
Knight, In: Viral and Mycoplasmal Infections of the Respiratory Tract, Lea & Febiger, Philadelphia, 1973.
Korutla and Kumar, Biochim. Biophys. Acta, 1224:597-600, 1994.
Korutla et al., Carcinogenesis, 16:1741-1745, 1995.
Koshkina et al., Cancer Chemother. Pharmacol., 47:451-456, 2001.
Koshkina et al., Cancer Chemotherapy Pharmacol 44: 187-192, 1999.
Koshkina et al., Journal Aerosol Medicine 17:7-14, 2004.
Kumar et al., Biochem. Pharmacol., 55:775-783, 1998.
Kumar et al., Neoplasia, 5(3):255-260, 2003.
Kumar et al., Nucleic Acids Symp. Ser., (44):75-76, 2000.
Kuo et al., Biochim. Biphys. Acta, 1317:95-100, 1996.
Kuttan et al., Cancer Lett., 29(2):197-202, 1985.
Li et al., Proc. Natl. Acad. Sci. USA, 90:1839-1842, 1993.
Lim et al., J. Neurosci., 21:8370-8377,2001.
Limtrakul et al., Cancer Lett., 116:197-203, 1997.
Lu et al., Carcinogenesis, 15:2363-2370,1994.
Mehta et al., Anticancer Drugs, 8:470-481, 1997.
Mishra et al., Nucleic Acids Res. Suppl., (2):277-278, 2002.
Mohan et al., J. Biol. Chem., 275:10405-10412,2000.
Mukhopadhyay et al., Agents Actions, 12(4):508-515, 1982.
Mukhopadhyay et al., Oncogene, 21(57):8852-8861, 2002.
Naidu and Thippeswamy, Mol. Cell Biochem., 229:19-23, 2002.
Naito et al., J. Atheroscler. Thromb., 9(5):243-250, 2002.
Natarajan and Bright, J. Immunol., 168:6506-6513, 2002.
New, In: Liposomes, a Practical Approach, Liverpool School of Tropical Medicine, Pembroke Place, Liverpool, 1990.
Nirmala and Puvanakrishnan, Biochem. Pharmacol., 51:47-51, 1996.
Nirmala and Puvanakrishnan, Mol. Cell Biochem., 159:85-93, 1996.
Nirmala et al., Mol. Cell Biochem., 197:31-37, 1999.
Ohtsu et al., J. Med. Chem., 7:45(23):5037-5042, 2002.
Osawa et al., Biosci. Biotechnol. Biochem., 59(9):1609-1612, 1995.
Pahl, Oncogene, 18:6853-6866, 1999.
Pan et al., Biochem. Pharmacol., 60:1665-1676, 2000.
Piwocka et al., Ann NY Acad. Sci, 973:250-254, 2002.
Piwocka et al., Exp. Cell Res., 249:299-307, 1999.
Plummer et al., Oncogene, 18:6013-6020, 1999.
Punithavathi et al., Br. J. Pharmacol., 131:169-172, 2000.
Quiles et al., Biofactors, 8:51-57, 1998.
Ramachandran and You, Breast Cancer Res. Treat., 54:269-278, 1999.
Ramirez-Tortosa et al., Atherosclerosis, 147:371-378, 1999.
Ranjan et al., J. Surg. Res., 87:1-5, 1999.
Rao et al., Cancer Res., 55:259-266, 1995.
Rao et al., Indian J. Med. Res., 75:574-578, 1982.
Rao et al., Indian J. Physiol. Pharmacol., 28(3):211-215, 1984.
Rasmussen et al., Planta Med., 66(4):396-398, 2000.
Ruby et al., Cancer Lett., 94(1):79-83, 1995.
Sidhu et al., Wound Repair Regen., 6:167-177, 1998.
Simon et al., Cancer Lett., 129:111-116, 1998.
Singh et al., Carcinogenesis, 19:1357-1360, 1998.
Soni and Kuttan, Indian J. Physiol. Pharmacol., 36:273-275, 1992.
Srivastava et al., Arzneimittelforschung, 36:715-717, 1986.
Srivastava et al., Protaglandins Leukot Essent Fatty Acids, 52:223-227, 1995.
Srivastava et al., Throm. Res., 40:413-417, 1985.
Sugiyama et al., Biochem. Pharmacol., 52(4):519-525, 1996.
Syu et al., J. Nat. Prod., 61(12):1531-1534, 1998.
Thaloor et al., Am. J. Physiol., 277:C320-329, 1999.
Tonnesen and Karlsen, Z Lebensm Unters Forsch., 180(5):402-404, 1985.
Venkatesan and Chandrakasan, Mol. Cell Biochem., 142:79-87, 1995.
Venkatesan et al., Life Sci., 61:P51-58, 1997.
Venkatesan, Br. J. Pharmacol., 124:425-427, 1998.
Venkatesan, Life Sci., 66:PL21-28, 2000.
Verschraegen, Clin. Cancer Research 10:2319-2326, 2004
Waldrep et al., J Aerosol Med., 7(2):135-145, 1994.
Weibel, In: Morphometry of the Human Lung, Academic Press, NY, 85, 1963.
Zhang et al., Carcinogenesis, 20:445-451, 1999.

1. A pharmaceutical lipid vehicle composition suitable for aerosol delivery to a subject, the composition comprising curcumin, one or more lipids, and an aqueous solvent, wherein:
(a) the transition temperature of the lipid, if only one lipid is present, or mean transition temperature of the lipids, if more than one lipid is present, is less than about 15° C., and
(b) the lipid vehicle composition can be nebulized.

2. The pharmaceutical composition of claim 1, wherein said composition comprises curcumin and one or more lipids dispersed as particles in an aqueous solvent.

3. The pharmaceutical composition of claim 1, wherein one or more of the lipids is a phospholipid.

4. The pharmaceutical composition of claim 1, wherein said composition comprises liposomes.

5. The pharmaceutical composition of claim 4, wherein a substantial portion of the liposomes can be nebulized without fracturing.

6. The pharmaceutical composition of claim 5, wherein greater than about 50% of the liposomes can be nebulized without fracturing.

7. The pharmaceutical composition of claim 6, wherein greater than about 75% of the liposomes can be nebulized without fracturing.

8. The pharmaceutical composition of claim 7, wherein greater than about 90% of the liposomes can be nebulized without fracturing.

9. The pharmaceutical composition of claim 5, wherein the liposomes form a complex with curcumin.

10. The pharmaceutical composition of claim 4, wherein the liposomes are unilamellar liposomes.

11. The pharmaceutical composition of claim 4, wherein the liposomes are multilamellar liposomes.

12. The pharmaceutical composition of claim 4, wherein the liposomes have an average mass median aerodynamic diameter of about 1 to about 3 microns after being nebulized.

13. The pharmaceutical composition of claim 1, wherein one or more of the lipids comprises a fatty acid moiety that is fully saturated.

14. The pharmaceutical composition of claim 1, wherein one or more of the lipids comprises a 12-carbon fatty acid moiety.

15. The pharmaceutical composition of claim 14, wherein one or more of the lipids comprises two 12-carbon fatty acid moieties.

16. The pharmaceutical composition of claim 1, comprising dilaurylphosphatidylcholine.

17. The pharmaceutical composition of claim 1, wherein one or more of the lipids comprises a monounsaturated fatty acid moiety.

18. The pharmaceutical composition of claim 1, wherein the transition temperature of the lipid or mean transition temperature of the lipids is less than about 5° C.

19. The pharmaceutical composition of claim 1, wherein the transition temperature of the lipid or mean transition temperature of the lipids is less than about 0° C.

20. The pharmaceutical composition of claim 1, wherein the transition temperature of the lipid or mean transition temperature of the lipids is greater than about −5° C.

21. The pharmaceutical composition of claim 1, further defined as comprising one or more anti-hyperproliferative agents.

22. The pharmaceutical composition of claim 21, wherein the anti-hyperproliferative agents are chemotherapeutic agents.

23. The pharmaceutical composition of claim 22, wherein the chemotherapeutic agents are doxorubicin, 5-fluorouracil, cisplatin, taxol, gemcitabin, BCNU, or camptothecin.

24. A method of treating a pathological condition in subject, comprising:
(a) providing a lipid vehicle composition comprising curcumin, one or more lipids, and an aqueous solvent, wherein the transition temperature of the lipid or mean transition temperature of the lipids is less than about 15° C., and wherein the lipid vehicle composition can be nebulized; and
(b) administering the composition to the subject by inhalation.

25. The method of claim 24, wherein the subject is a human subject.

26. The method of claim 24, further comprising having the subject inhale 5% CO₂ either before or during administration of the pharmaceutical composition to the subject.

27. The method of claim 24, wherein said composition comprises curcumin and one or more lipids dispersed as particles in an aqueous solvent.

28. The method of claim 24, wherein one or more of the lipids is a phospholipid.

29. The method of claim 24, wherein said composition comprises liposomes.

30. The method of claim 29, wherein a substantial portion of the liposomes can be nebulized without fracturing.

31. The method of claim 30, wherein greater than about 50% of the liposomes can be nebulized without fracturing.

32. The method of claim 31, wherein greater than about 75% of the liposomes can be nebulized without fracturing.

33. The method of claim 32, wherein greater than about 90% of the liposomes can be nebulized without fracturing.

34. The method of claim 29, wherein the liposomes form a complex with curcumin.

35. The method of claim 29, wherein the liposomes are unilamellar liposomes.

36. The method of claim 29, wherein the liposomes are multilamellar liposomes.

37. The method of claim 29, wherein the liposomes have an average mass median aerodynamic diameter of about 1 to about 3 microns after being nebulized.

38. The method of claim 24, wherein one or more of the lipids comprises a fatty acid moiety that is fully saturated.

39. The method of claim 24, wherein one or more of the lipids comprises a 12-carbon fatty acid moiety.

40. The method of claim 39, wherein one or more of the lipids comprises two 12-carbon fatty acid moieties.

41. The method of claim 24, comprising dilaurylphosphatidylcholine.

42. The method of claim 24, wherein one or more of the lipids comprises a monounsaturated fatty acid moiety.

43. The method of claim 24, wherein the transition temperature of the lipid or mean transition temperature of the lipids is less than about 5° C.

44. The method of claim 43, wherein the transition temperature of the lipid or mean transition temperature of the lipids is less than about 0° C.

45. The method of claim 44, wherein the transition temperature of the lipid or mean transition temperature of the lipids is greater than about −5° C.

46. The method of claim 24, wherein composition is administered to the subject using a nebulizer.

47. The method of claim 24, further defined as a method of treating a hyperproliferative disease in a subject.

48. The method of claim 47, wherein the hyperproliferative disease is cancer.

49. The method of claim 48, wherein the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, leukemia, or multiple myeloma.

50. The method of claim 47, further comprising administering to the subject a therapeutically effective amount of a secondary anti-hyperproliferative agent.

51. The method of claim 50, wherein the secondary anti-hyperproliferative agent is a chemotherapeutic agent.

52. The method of claim 51, wherein the chemotherapeutic agent is doxorubicin, daunorubicin, mitomycin, actinomycin D, bleomycin, cisplatin, VP16, an enedyine, taxol, vincristine, vinblastine, carmustine, melphalan, cyclophosphamide, chlorambucil, busulfan, lomustine, 5-fluorouracil, gemcitabin, BCNU, or camptothecin.

53. The method of claim 24, further defined as a method of treating hypercholesterolemia.

54. The method of claim 24, further defined as a method of promoting wound healing.

55. The method of claim 24, further defined as a method of preventing skin wrinkling.

56. The method of claim 24, further defined as a method of treating inflammation.

57. The method of claim 56, wherein the inflammation is pulmonary inflammation.

58. The method of claim 57, wherein the pulmonary inflammation is pulmonary inflammation secondary to asthma.

59. The method of claim 24, further defined as a method of treating arthritis.

60. The method of claim 59, further defined as a method of treating rheumatoid arthritis.

61. The method of claim 24, further defined as a method of treating viral infection.

62. The method of claim 61, wherein the viral infection is HIV infection.

63. The method of claim 24, further defined as a method of treating pulmonary disease.

64. The method of claim 24, further defined as a method of treating a disease associated with abnormal immune function.

65. The method of claim 64, wherein the disease associated with abnormal immune function is cerebrodegenerative disease.

66. The method of claim 65, wherein the cerebrodegenerative disease is multiple sclerosis.

67. The method of claim 24, further defined as a method of treating diabetes.

68. The method of claim 24, further defined as a method of treating myocardial ischemia.
